(19) United States
(12) Patent Application Publication
(10) Pub. No.: US 2018/0304086 A1
(43) Pub. Date: Oct. 25, 2018

(54) WIRELESS COMMUNICATION FRAME DESIGN FOR MEDICAL IMPLANTS

(71) Applicant: QUALCOMM Incorporated, San Diego, CA (US)

(72) Inventors: Stephen Jay Shellhammer, Ramona, CA (US); Osvaldo Alcala, Chula Vista, CA (US); Linhai He, San Diego, CA (US)

(21) Appl. No.: 15/707,379

(22) Filed: Sep. 18, 2017

Related U.S. Application Data
(60) Provisional application No. 62/487,443, filed on Apr. 19, 2017.

(51) Int. Cl. A61N 1/372; A61B 5/00; H04B 7/06; H04B 1/717

(52) U.S. Cl. CPC A61N 1/37223; A61B 5/0031; H04B 1/7174; A61B 5/6847; A61B 5/7221; H04B 7/0608

(57) ABSTRACT

Techniques provided herein are directed toward providing a robust downlink communication frame that enables medical implants with highly inaccurate LOs to reliably provide uplink communications to an interrogator device. The downlink communication frame can include, among other things, a plurality of uplink trigger subframes that enable timing of uplink communication of the various medical implants with which the interrogator device is communicating. These uplink trigger subframes may be modulated in a special manner as to distinguish them from other subframes.

### RELATED APPLICATIONS

This application claims the benefit of U.S. Provisional Application No. 62/487,443, filed Apr. 19, 2017, entitled “WIRELESS COMMUNICATION FRAME DESIGN FOR MEDICAL IMPLANTS”, which is assigned to the assignee hereof, and incorporated by reference herein in its entirety.

### BACKGROUND

A wireless medical implant system for a patient can comprise an interrogator device, typically in, on, or in proximity to the patient, and a plurality of electronic medical implants that can take biological measurements of a body part (e.g., biological tissue) and communicate them to the interrogator device. The interrogator device can then communicate this information to other devices, such as a mobile phone, tablet, or medical device of the patient or patient's healthcare provider. The interrogator device can also communicate with the medical implants to cause them to stimulate the body part.

However, the medical implants may need to operate on very low power consumption. This can provide severe power constraints on the design of the local oscillator used for wireless communication. Thus, to preserve power in a medical implant system, wireless communication may need to tolerate relatively large inaccuracies in the carrier frequency. In addition, these medical implants may be very small and may not have physical space to include a crystal, for a crystal oscillator, which is another reason the local oscillator frequency may be relatively inaccurate.

### SUMMARY

Techniques provided herein are directed toward providing a robust downlink communication frame that enables medical implants with highly inaccurate LOs to reliably provide uplink communications to an interrogator device. The downlink communication frame can include, among other things, a plurality of uplink trigger subframes (or fields) that enable timing of uplink communication of the various medical implants with which the interrogator device is communicating. These uplink trigger subframes may be modulated in a special manner as to distinguish them from other subframes.

A medical device, according to the disclosure, comprises a communication interface configured to receive a communication frame transmitted via a radio frequency (RF) signal and comprising a synchronization subframe, a payload subframe, and a plurality of uplink trigger subframes spaced apart such that at least one uplink subframe can be transmitted between successive uplink trigger subframes. The medical device further comprises a processing unit communicatively coupled with the communication interface and configured to determine when to send an uplink subframe via the communication interface based, at least in part, on when at least one of the plurality of uplink trigger subframes was received, and send, via the communication interface, the uplink subframe.

The medical device can comprise one or more the following features. The processing unit may be configured to determine when to send the uplink subframe by counting a number of uplink trigger subframes of the plurality of uplink trigger subframes, by obtaining an identifier from the at least one of the plurality of uplink trigger subframes, or by adjusting the local clock based on when the at least one of the plurality of uplink trigger subframes was received. The processing unit may be further configured to identify the at least one of the plurality of uplink trigger subframes by analyzing a pair of pulses in the at least one of the plurality of uplink trigger subframes, wherein the pair of pulses is modulated such that a first pulse has a first duration, and a second pulse has a second duration different than the first duration. The processing unit may be configured to analyze the pair of pulses in the at least one of the plurality of uplink trigger subframes by comparing a ratio of the first duration to the second duration. The processing unit may be configured to send the uplink subframe between successive uplink trigger subframes of the plurality of uplink trigger subframes.

An interrogator device, according to the description, comprises a processing unit configured to generate a communication frame comprising a synchronization subframe, a payload subframe, and a plurality of uplink trigger subframes. The interrogator device further comprises a communication interface communicatively coupled with the processing unit and configured to send the communication frame via a radio frequency (RF) signal, and receive at least one uplink frame between successive uplink trigger subframes in the communication frame.

The interrogator device may comprise one or more of the following features. The processing unit may be further configured to include, in each uplink trigger subframe of the plurality of uplink trigger subframes, an identifier unique to the respective uplink trigger subframe. The processing unit may be further configured to include in each uplink trigger subframe of the plurality of uplink trigger subframe a first pair of pulses, wherein the first pair of pulses uses a first modulation scheme such that a first pulse has a first duration, and a second pulse has a second duration different than the first duration. The processing unit may be further configured to cause a ratio of the first duration to the second duration to exceed a threshold. The processing unit may be configured to use a second modulation scheme to modulate a second pair of pulses in the payload subframe, wherein the second modulation scheme is different than the first modulation scheme.

An example method of synchronizing wireless communication at a medical device, according to the description, comprises receiving, at the medical device, a communication frame transmitted via a radio frequency (RF) signal and comprising a synchronization subframe, a payload subframe, and a plurality of uplink trigger subframes spaced apart such that at least one uplink subframe can be transmitted between successive uplink trigger subframes. The method further comprises determining, with the medical device, when to send an uplink subframe based, at least in part, on when at least one of the plurality of uplink trigger subframes was received, and sending, with the medical device, the uplink subframe.

The method can further comprise one or more of the following features. Determining when to send the uplink subframe may comprise counting a number of uplink trigger subframes of the plurality of uplink trigger subframes, obtaining an identifier from the at least one of the plurality of uplink trigger subframes, or adjusting a local clock of the medical device based on when the at least one of the plurality of uplink trigger subframes was received. The method may further comprise identifying the at least one of the plurality of uplink trigger subframes by analyzing a pair of pulses in the at least one of the plurality of uplink trigger subframes, wherein the pair of pulses is modulated such that a first pulse has a first duration, and a second pulse has a second duration different than the first duration. Analyzing the pair of pulses in the at least one of the plurality of uplink trigger subframes comprises comparing a ratio of the first duration to the second duration. Sending the uplink subframe may comprise sending the uplink subframe between successive uplink trigger subframes of the plurality of uplink trigger subframes.

A method of enabling synchronized wireless communication with a interrogator device, according to the description, comprises generating, with the interrogator device, a communication frame comprising a synchronization subframe, a payload subframe, and a plurality of uplink trigger subframes. The method further comprises sending, with the interrogator device, the communication frame via a radio frequency (RF) signal, and receiving, at the interrogator device, at least one uplink frame between successive uplink trigger subframes in the communication frame.

The method may further comprise one or more of the following features. The method may comprise including, in each uplink trigger subframe of the plurality of uplink trigger subframes, an identifier unique to the respective uplink trigger subframe. The method may comprise including in each uplink trigger subframe of the plurality of uplink trigger subframe a first pair of pulses, wherein the first pair of pulses uses a first modulation scheme such that a first pulse has a first duration, and a second pulse has a second duration different than the first duration. The method may comprise causing a ratio of the first duration to the second duration to exceed a threshold. The first modulation scheme may be different than a second modulation scheme used to modulate a second pair of pulses in the payload subframe.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is a simplified cross-sectional diagram illustrating an embodiment of a wireless medical implant system.

FIG. 2A is an illustration of how calibration frames and can be communicated and what they may comprise, according to some embodiments.

FIG. 2B is a diagram of a pulse pair that illustrates how signals carrying the calibration frames may be modulated, according to an embodiment.

FIG. 3 is an illustration of how operating frames can be communicated and what they may comprise, according to some embodiments.

FIG. 4 is an illustration of example pulse pair that can be used in an uplink trigger subframe.

FIG. 5 is an illustration of example contents of an uplink subframe, according to an embodiment.

FIG. 6 is a flow diagram of an example method of synchronizing wireless communication at a medical device, according to an embodiment.

FIG. 7 is a flow diagram of an example method of enabling synchronize wireless communication with an interrogator device, according to an embodiment.

FIG. 8 is a simplified block diagram of an interrogator device, according to an embodiment.

FIG. 9 is a simplified block diagram of a medical implant, according to an embodiment.

Elements, stages, steps, and actions in the figures with the same reference label in different drawings may correspond to one another (e.g., may be similar or identical to one another). Further, some elements in the various drawings are labelled using a numeric prefix followed by a numeric suffix (where the numeric prefix and the numeric suffix are separated by a hyphen). Elements with the same numeric prefix but different suffices may be different instances of the same type of element. The numeric prefix without any suffix is used herein to reference any element with this numeric prefix.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It will be understood by a person of ordinary skill in the art that, although the embodiments provided herein are directed toward medical applications, the techniques described herein may be utilized in other applications involving digital communication. Additionally, embodiments provided herein describe the use of “medical implants,” although such implants may be utilized to gather data and/or stimulate a body part without necessarily performing a medical function. Moreover, and indicated below, embodiments may utilize medical devices that may or may not be partially or wholly implanted or implantable. A person of ordinary skill in the art will recognize many variations.

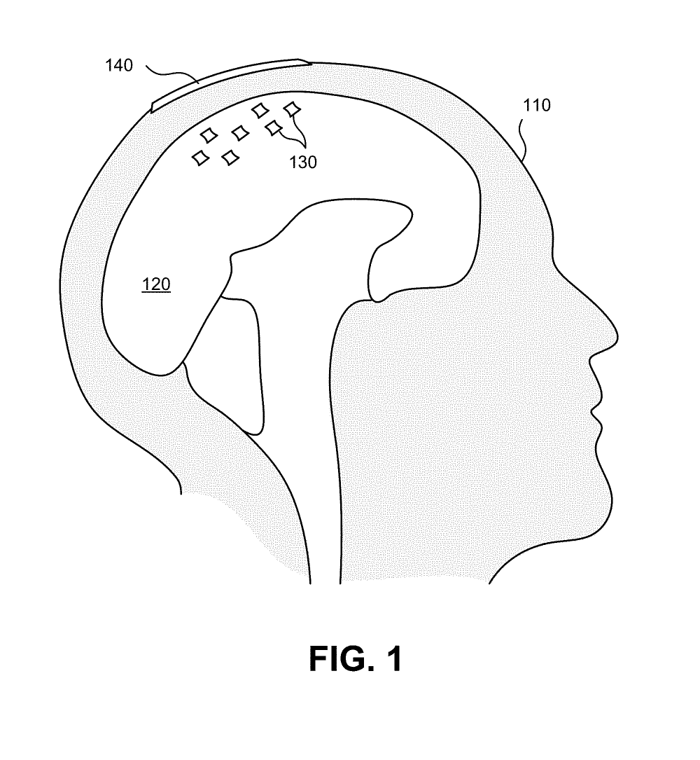

FIG. 1 is a simplified cross-sectional diagram illustrating an embodiment of a wireless medical implant system. Here, a patient's head 110 is illustrated, indicating a portion of the brain 120 in which a plurality of medical implants 130 are implanted. (For clarity, only a portion of the medical implants are labeled.) An interrogator device 140 uses low-power, short-range radio frequency (RF) signals at a designated frequency not only to communicate with the one or more medical implants, but also, in some embodiments, to provide power to the implants. Such wireless communication can employ any of a variety of short-range wireless technologies, including near-field communication (NFC) and/or other wireless technologies. According to some embodiments, data may be communicated in a secure fashion (e.g., using any of a variety of encryption techniques).

For scenarios in which the wireless medical implant system is utilized to measure and stimulate a portion of the brain (as shown in FIG. 1), the interrogator device 140 may be referred to as a “skin patch” because it may be substantially flat in shape and may be disposed on or near the patient's skin. The medical implants 130 in such scenarios may be referred to as “neurograins” because of their relatively small size and location within the patient's brain. A person of ordinary skill in the art will appreciate that alternative embodiments may have medical implants located in one or more other areas (other than the brain) of a patient's body and/or in or other wireless devices non-medical wireless communication applications. Moreover, for medical applications, interrogator device 140 may comprise one or more physical units located in, on and/or in proximity to the patient's body.

Depending on the application, the wireless medical implant system may comprise hundreds or thousands of medical implants 130. (Alternative embodiments may include a smaller or larger number of medical implants 130 than this.) These medical implants 130 can also communicate back to the interrogator device 140 (e.g., through RF backscatter, by changing the impedance of their respective antennas) using, for example, a time division multiple access (TDMA) protocol. The interrogator device 140 may coordinate the uplink transmission.

Medical implants 130 can comprise active devices (having a power source) and/or passive devices (having no power source) configured to take biological measurements of the brain 120 (e.g., information regarding electrical signals generated by the patient's brain cells) and communicate the measurements to the interrogator device 140 and/or provide stimulation of the patient's brain 120 (e.g., via one or more electrodes), where such stimulation may be based on communication received from the interrogator device 140. In some embodiments, active medical implants may also draw power wirelessly from the interrogator device, which may be used to charge their batteries (or other power sources), or the implant may work directly off the wireless power without having a battery. As previously noted, medical implants 130 can be powered by the interrogator device 140 using, for example, a coiled antenna drawing power from communications and/or other signals or fields generated by the interrogator device 140. It can be noted that, in alternative embodiments, the interrogator device 140 may comprise multiple antennas, and/or the biological measurement and stimulation system may have one or more nodes and/or devices between the medical implants 130 and the interrogator device 140. Because medical implants 130 can vary in functionality, they can vary in size, shape, type, and/or may have electrodes (and or other sensors) that vary as well.

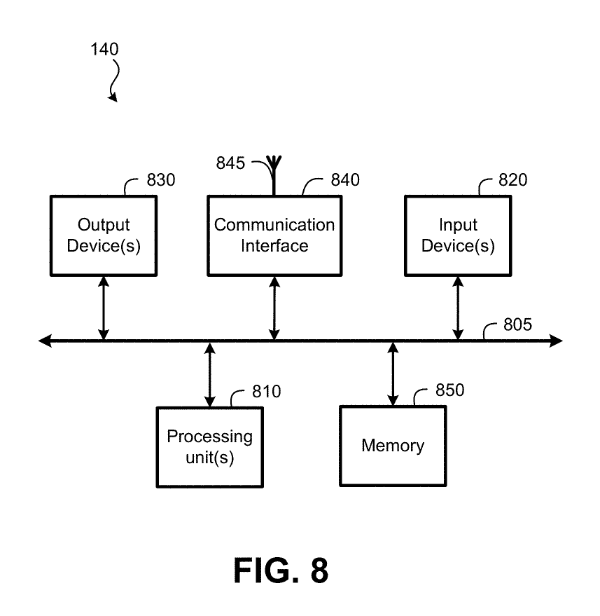

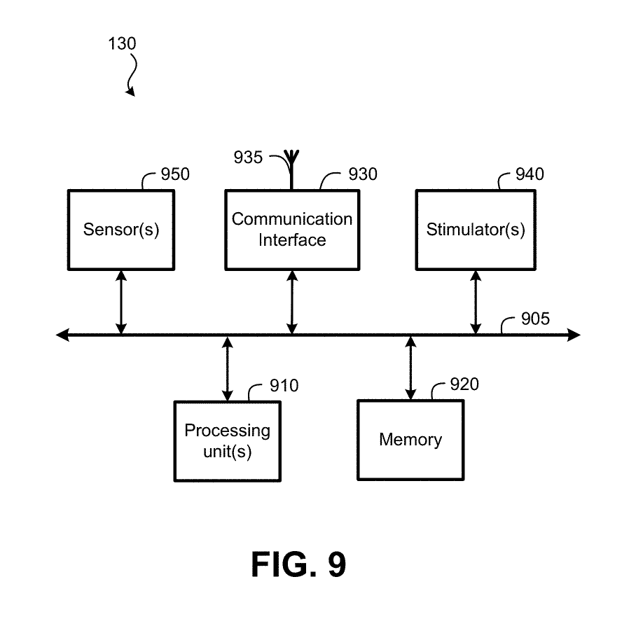

A person of ordinary skill in the art will appreciate the basic hardware configuration of an interrogator device 140 and/or medical implant 130. This can include, for example, a power source, processing unit, communication bus, volatile and/or non-volatile memory (which may comprise a non-transitory computer-readable medium having computer code for execution by the processing unit), transceiver, antenna, etc. The medical implant 130 may further comprise one or more sensors, electrodes, and/or stimulators utilized for sensing and/or stimulating one or more parts of the body. As such, the interrogator device 140 and/or medical implant 130 may have means for performing some, or all, of the functions described herein using one or more of its hardware and/or software components. In some embodiments, components may be selected and/or optimized for low power consumption. In particular, because medical implants 130 may be limited in size and/or power, the medical implants 130 may not have the same memory size and/or processing capabilities as the interrogator device 140. Example electrical hardware and software components of an interrogator device 140 and medical implant 130 are illustrated in FIG. 8 and FIG. 9, respectively, and described in more detail below.

As noted above, the medical implants 130 may be passive or active implants that collect energy from the interrogator device 140, and thereby may need to operate on very low power consumption. But this can provide severe power constraints on the design of the local oscillator (LO) used for RF communication, because the accuracy of the LO is directly related to power consumption: low-power LOs are generally less accurate than relatively higher-power LOs. In addition, due to size constraints there is insufficient area to include a crystal on the medical device, making it more difficult to produce an accurate LO frequency. Thus, to preserve power in a medical implant system, wireless communication may need to tolerate relatively large inaccuracies in the carrier frequency. (Because an interrogator device is typically given a larger power budget than the medical implant, it may have a more accurate LO then the medical implant. The medical implant, however, may have a highly inaccurate LO.)

For example, in some embodiments, the LO of the medical implants (which may comprise a simple ring oscillator) can vary in frequency accuracy from approximately ±10% to approximately ±30%. Other embodiments may experience larger or smaller frequency inaccuracies. However, downlink communications from the interrogator device to the medical implants occurs at a low data rate, using, for example, only three bits per medical implant per frame, though the number of bits transmitted to each medical implant may be more or less than three bits per frame

Traditional implementations of a wireless medical implant system attempted to overcome issues that arise with inaccurate medical implant LOs by removing them altogether and instead deriving a local clock of the medical implant from two RF carriers transmitted by the interrogator device. Although this design facilitates synchronization, it results in significant interference in wireless medical implant systems where many medical implants are utilized.

To avoid this interference, embodiments provided herein may be utilized by a wireless medical implant systems within interrogator device having a single RF carrier and each medical implant having its own LO to generate its local clock. Interference may be reduced, at least in part, since the LOs of the various medical implants will not be in phase, so interference caused by non-transmitting operations performed by the medical implants is reduced, being spread over time. However, as noted previously, the LO of the medical implants can be highly inaccurate.

Techniques disclosed herein are directed toward providing a robust downlink communication frame in a wireless medical implant system where downlink communication from an interrogator device of the wireless medical implant system to one or more medical implants may be impacted by the inaccuracy of the LO of the medical implants. In particular, the downlink communication frame can include, according to embodiments, a plurality of uplink trigger subframes that enable timing of uplink communication of the various medical implants with which the interrogator device is communicating. These uplink trigger subframes may be modulated in a special manner as to distinguish them from other subframes.

The downlink communication frame having uplink trigger subframes may comprise an operating frame, in which operating data is communicated to and/or from medical implants 130. Optionally, however, downlink communication may additionally include a calibration frame used to help calibrate LOs of medical implants.

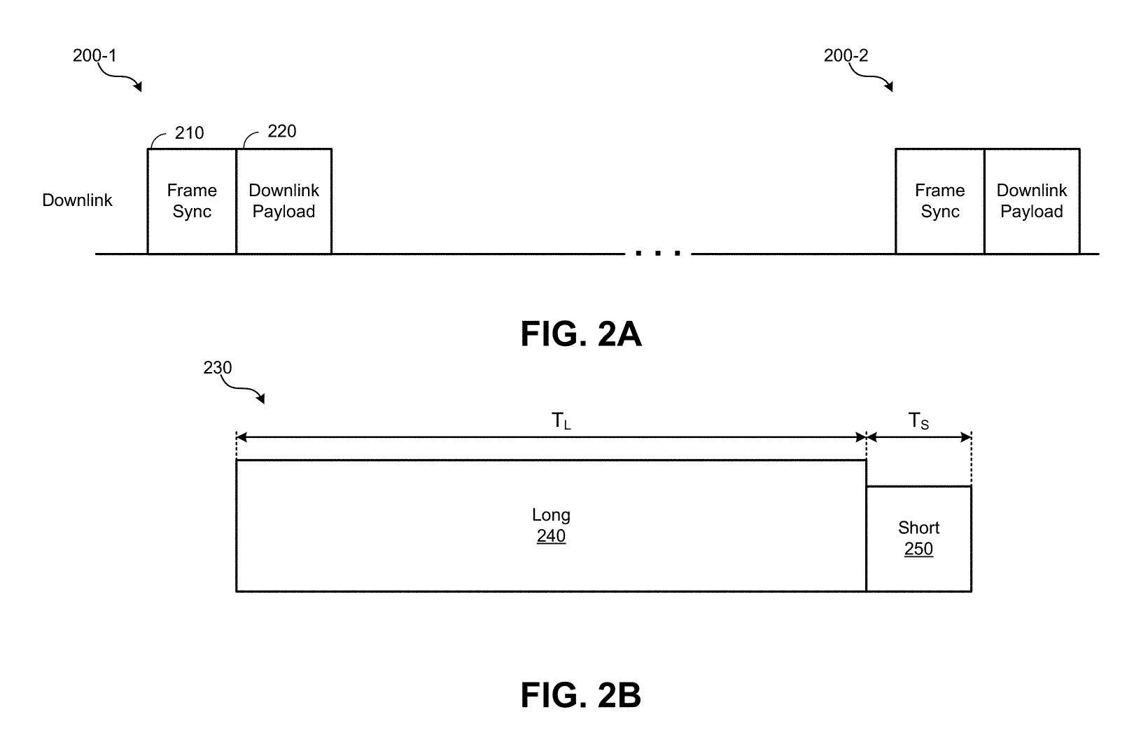

FIG. 2A is an illustration of how calibration frames 200-1 and 200-2 (collectively and generically referred to herein as calibration frames 200) can be communicated and what they may comprise, according to some embodiments. When the wireless medical implant system is first activated and the medical implants are first powered on, the LO frequencies of the various medical implants may vary significantly, as noted above. These calibration frames 200 provide one way in which the accuracy of the LOs may be improved.

Generally speaking, a calibration frame 200 is a downlink frame (sent from the interrogator device to the medical implants) with signals identifiable by the medical implants as calibration signals, enabling the medical implants to calibrate their LOs. Put briefly, the calibration frame 200 may include only downlink information (without waiting for an uplink response), and may be modulated differently to identify it as a calibration frame. Additional details regarding calibration are provided herein below as well as U.S. Pat. App. No. 62/480,945 entitled “Pulse Width Modulated Amplitude Modulation” which is hereby incorporated by reference in its entirety for all purposes. (This application referred to herein below as “the '945 application”.) This calibration can improve the accuracy of the LOs to approximately ±200 parts per million (ppm). In alternative embodiments where laser trimming or calibration is performed during the manufacturing of the medical implants (which may reduce the frequency variation in the LOs to less than 1%), a calibration frame may not be needed. As illustrated, calibration frames 200 may be repeated any number of times (depending on desired functionality), if desired, to help ensure calibration of the medical implants.

As illustrated, the calibration frame comprises a frame sync subframe 210 to allow medical implants to find the beginning of the calibration frame 200. A downlink payload subframe 220 that (in the case of the calibration frame 200) carries information indicating that it is a calibration frame. Following the downlink payload subframe 220, there may be a relatively long period in which there is no downlink data (but the RF carrier may still be transmitted by the interrogator device to power the medical implants).

FIG. 2B is a diagram of a pulse pair 230 that illustrates how signals carrying the calibration frames 200 may be modulated, according to an embodiment. As detailed in the '945 application, a digital bit of information may be encoded in a pair of pulses having different amplitudes. For example, each bit may comprise a first pulse with a “high” amplitude and a second pulse with a “low” amplitude. (This is, however, chosen arbitrarily. Alternative embodiments may choose to have a “low” first pulse and a “high” second pulse. Additionally, the amplitude of the “high” and “low” pulses may vary, as detailed in the '945 application.) In the example, a frame sync subframe 210 an include the pulse pair 230, which comprises a “high” pulse 240 many times longer than the “low” pulse 250 that follows. In some embodiments, for example, the “high” pulse 240 may be six times longer than the “low” pulse 250. (Of course, the ratio between long and short pulses may be different in alternative embodiments.) Here, the length of the long “high” pulse in the frame sync subframe 210 identifies the frame sync subframe as a frame sync subframe to the medical devices. Additional information included in the calibration frame 200 (e.g., in the downlink payload subframe 220) may be encoded in a similar manner, where the value of a digital bit is based on which pulse in a pulse pair is longer (the first, “high” pulse or the second, “low” pulse), and where the ratio between long and short pulses is distinguishable over the pulses in the frame sync subframe (using, for example, a different ratio of pulse length between long and short pulses). Again, additional details are provided in the '945 application.

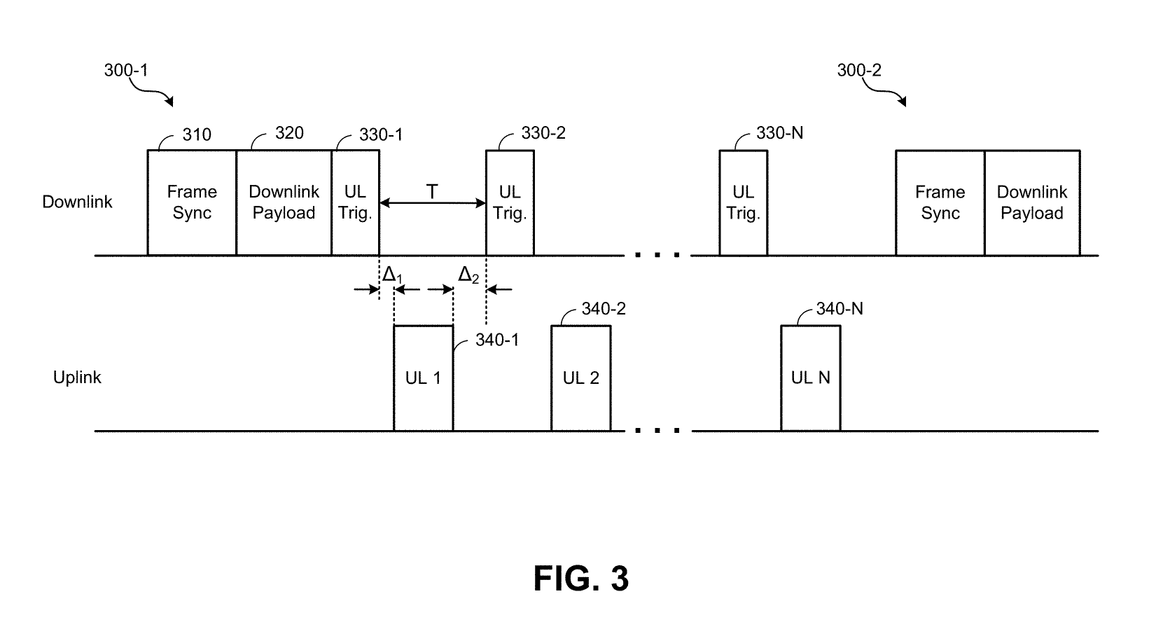

FIG. 3 is an illustration of how operating frames 300-1 and 300-2 (collectively and generically referred to herein as operating frames 300) can be communicated and what they may comprise, according to some embodiments. As illustrated, operating frames 300 can include both downlink (interrogator device to medical implant) and uplink (medical implant to interrogator device) communications. Because the medical implants utilize backscatter, the interrogator device may send an RF carrier during the entire operating frame, enabling the medical implants to backscatter the RF carrier for uplink communications to the interrogator device.

As illustrated, similar to the calibration frame 200 of FIG. 2A, operating frames 300 can include a frame sync subframe 310 and downlink payload 320. Here, the downlink payload 320 can include information for the medical implants to, for example, control stimulation (turning it on/off, communicating a level of stimulation, etc.), brain function sensing, and/or other functionality. Again, and data can be encoded in pulse pairs, as previously described.

In the operating frame, additional uplink trigger subframes 330-1, 330-2, and 330-N (collectively and generically referred to herein as uplink trigger subframes 330) are also included, to help ensure the medical implants respond when scheduled. As illustrated in FIG. 3, the downlink communication sent by the interrogator device includes the frame sync subframe 310, downlink payload subframe 320, and uplink trigger subframes 330 followed by time slots for each of the medical implants to provide, in turn, uplink communications.

In a given operating frame, there may be a set number of slots, N, for medical implants to communicate. In some embodiments, for example, there may be 1000 slots. Other embodiments may have a higher or lower number of slots, depending on desired functionality. In some embodiments, the number of slots may be dynamic. The number N may be determined by the interrogator device and conveyed to the medical implants in the downlink payload subframe 320, for example.

Because the LO of each medical implant may still vary (e.g., by ±200 ppm) even after calibration, there is a chance that the medical implants may not be able to keep time accurately enough to communicate in their designated slot if they perform local synchronization only once per operating frame. For example, by the time slot number 1000 occurs in the operating frame 300, the medical implant scheduled to communicate during that slot may have experienced enough clock drift from the beginning of the operating frame 300 to have caused it to have communicated already (perhaps interfering with another medical implants communications), or it may not communicate until after slot number 1000 occurs.

To help mitigate these types of errors, uplink trigger subframes 330 can be located throughout the operating frame 300 to help keep the medical implants synchronized. As illustrated, some embodiments may include an uplink trigger subframe 330 before each slot for uplink communication. (In other words, for an operating frame 300 having N slots for uplink communication, there will be N uplink trigger subframes 330.) Some embodiments may include an uplink trigger subframe before a group of two or more slots for uplink communication. (E.g., for each uplink trigger a group of medical implants can respond, one at a time, before the next uplink trigger. In other words, for an operating frame 300 having N slots for uplink communication, there may be M uplink trigger subframes 330, where M=N/2, N/3, or some other fraction of N, depending on desired functionality.). The use of uplink trigger subframes 330 therefore enables each medical implant to more accurately determine the passage of time during the operating frame 300 by identifying the uplink trigger subframes 330 (which are based on the LO of the interrogator device, which is far more accurate). In other words, medical devices can synchronize with each uplink trigger subframe 330 to help mitigate the effects of clock drift during the operation frame 300.

According to embodiments, the length of time between uplink trigger frames may be larger than the length of time for uplink communication, allowing for medical implants to respond to the uplink trigger subframe, and also allowing for clock error (expansion or compression of uplink transmission due to clock inaccuracy). For example, in cases where uplink trigger subframes are sent for each uplink slot (as illustrated), there may be a period of time, Δ₁, after the interrogator device completes the transmission of an uplink trigger subframe 330-1 and before a medical implant begins transmission of a corresponding uplink subframe 340-1 (labeled in FIG. 3 as “UL 1”). This period of time may be set based on a determined period of time it may take the interrogator device to change from a transmission mode (e.g., transmitting the uplink trigger subframe 330-1) to a receive mode (e.g., ready to receive the corresponding uplink subframe 340-1), and may allow for a certain (e.g., expected maximum) amount of clock error of the medical devices.

There may also be a second period of time, Δ₂, after the medical implant ends transmission of the corresponding uplink subframe 340-2 and before the interrogator device begins transmission of the subsequent uplink trigger subframe 330-2. Due to clock drift during the transmission of the corresponding uplink subframe 340- 1, the value of Δ₂ may be subject to more variance than the value of Δ₁. Accordingly, the interrogator device may take into account worst-case clock inaccuracies of the medical devices. That said, because clock error of the medical implants can be reduced to less than 1% once calibrated, the additional overhead needed to accommodate worst-case clock expansion remains minimal. The value of Δ₂ may also take into account a length of time it may take the interrogator device to change from a receive mode back to a transmission mode.

Ultimately, the interrogator device may determine a period of time, T, between the transmittal of uplink trigger subframes 330 by adding a maximum expected value Δ₁, a length of time of an uplink subframe 340, and a maximum expected value of Δ₂. As previously noted, the maximum expected value of Δ₂ can be based on worst-case clock expansion what the uplink trigger subframe 330, and therefore time T can be set to accommodate different amounts of clock error. Depending on the amount of clock error allowable, the calibration frame to reduce clock error may not be needed, in some embodiments.

In some embodiments, the uplink trigger subframes 330 may be identical. In this case, each medical implant may utilize a counter that counts the number of uplink trigger subframes 330 in an operating frame to ensure that it communicates in its designated uplink slot. For instance, when the value of the counter matches an address of the medical implant after counting the latest uplink trigger subframe 330, the medical implant may then send an uplink subframe 340 during the time slot following the uplink trigger subframe 330.

In some embodiments, each uplink trigger subframe 330 may include information such as a counter, medical device address, or other identifier to indicate its position in the operating frame 300 and/or indicate the medical device designated to provide an uplink subframe 340 in the following uplink slot. In this case, the medical implant may not need to include the additional hardware (and/or software) required to implement a counter.

As indicated in FIG. 3 with a second operating frame 300-2 following a first operating frame 300-1, operating frames 300 can repeat. In the subsequent operating frame(s), new frame sync and downlink payload subframes can be transmitted and communications communicating with the same medical implants or different medical implants as the first operating frame 300-1 can follow in a manner similar to that described above. (Where different operating frames 300 communicate with different sets of medical implants, the downlink payload subframe 320 of the operating frame 300 may indicate which subset of medical implants is selected for communication during the operating frame 300 with the interrogator device.) The total length of the operating frame can vary, depending on latency, number of medical implants, and/or other factors. In some embodiments, for example, the operating frame is about 100 ms. (In other embodiments, the operating frame may be longer or shorter.)

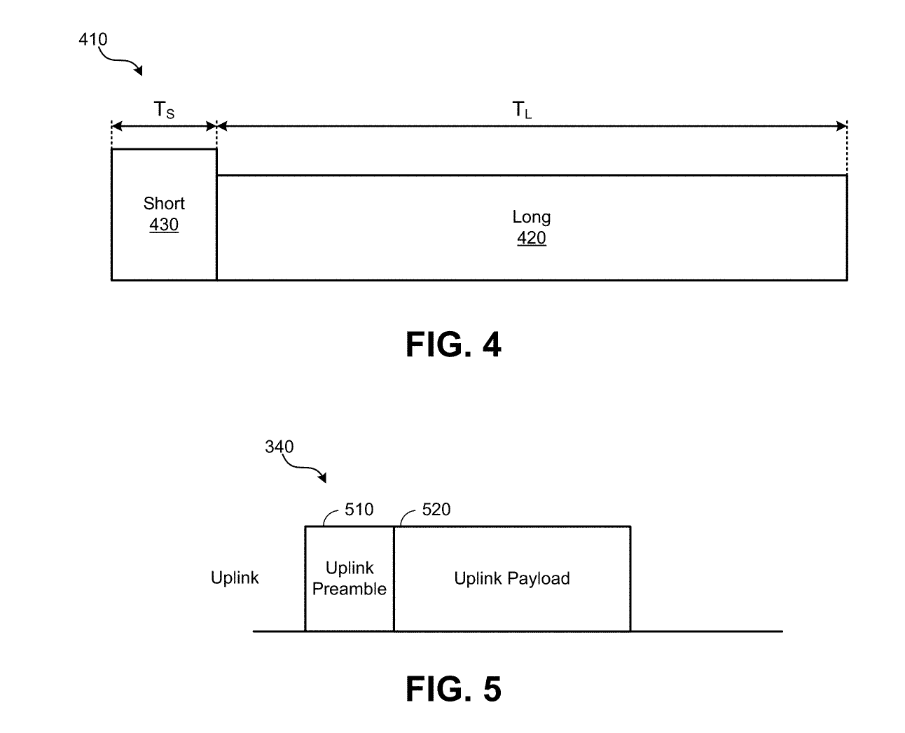

Similar to the frame sync subframes in calibration and operations frames described above, the uplink trigger subframe 340 can include digital bits comprising pulse pairs modulated differently than pulse pairs in other communications. FIG. 4 is an illustration of example pulse pair 410 that can be used in an uplink trigger subframe 340, to identify the uplink trigger subframe 340 as such. Similar to the frame sync subframes, the pulse pair 410 includes a long pulse 420 and a short pulse 430, where the ratio of the length of time of the long pulse 420 to the length of time of the short pulse 430 is especially high (thereby making it identifiable to medical devices). In this embodiment, however, the first (“high”) pulse is the short pulse 430, and the second (“low”) pulse is the long pulse 420, making the ordering of the short and long pulses opposite from that of the short and long pulses of the phrase sync subframes (as illustrated, for example, in FIG. 2B). Thus, although the ratio of long to short pulses may be similar, the medical devices can distinguish between the frame sync subframes and the uplink trigger subframes by determining the order of the long and short pulses in the pulse pairs.

FIG. 5 is an illustration of example contents of an uplink subframe 340, according to an embodiment. As previously indicated, each medical device can transmit and uplink subframe during its designated transmission slot, which can include data specific to that medical device. Here, the uplink subframe 340 can comprise an uplink preamble 510 followed by an uplink payload 520. The uplink preamble 510 can be used by the interrogator device to perform timing correction and, in some embodiments, may comprise a maximal length sequence. The uplink payload 520 carries data (e.g., operating status, sensor data, etc.) from the medical device to the interrogator device.

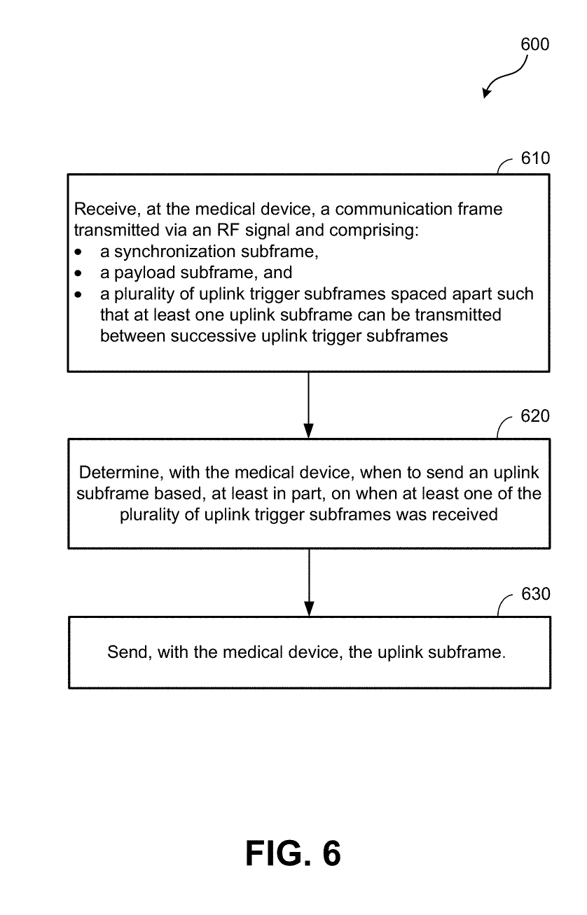

FIG. 6 is a flow diagram of an example method 600 of synchronizing wireless communication at a medical device, according to an embodiment. Here, the functionality described in one or more of the blocks illustrated in FIG. 6 may be performed, for example, by a medical device in a low-power wireless system, such as a medical implant 130 of the wireless medical device system illustrated in FIG. 1. Accordingly, means for performing this functionality may include hardware and/or software components of a medical implant 130. An example of such hardware and/or software components is illustrated in FIG. 9 and described in more detail below. Nonetheless, it will be understood that medical devices other than implanted or implantable medical devices may be utilized, in some embodiments.

The functionality at block 610 includes receiving, at the medical device, a communication frame transmitted via an RF signal and comprising a synchronization subframe, a payload subframe, and the plurality of uplink trigger subframes spaced apart such that at least one uplink subframe can be transmitted between successive uplink trigger subframes. Here, the communication frame may comprise an operating frame, as described in the embodiments above and illustrated in FIG. 3. As previously noted, the time between successive subframes (e.g., time T in FIG. 3) can allow for a buffer of time before and after the transmittal of an uplink subframe, and may accommodate expected worst-case clock drift for medical devices. Means for performing the functionality at block 610 may comprise, for example, bus 905, processing unit(s) 910, memory 920, communication interface 930, antenna 935, and/or other components of a medical implant 130, as illustrated in FIG. 9 and described in more detail below.

At block 620, the functionality comprises determining, with of the medical device, when to send an uplink subframe based, at least in part, on when at least one of the plurality of uplink trigger subframes was received. In some embodiments, determining when to send the uplink subframe may comprise counting a number of uplink trigger subframes of the plurality of uplink trigger subframes. In some embodiments, this may be done by a dedicated hardware counter of the medical device. Additionally or alternatively, in some embodiments, determining when to send the uplink subframe may comprise obtaining an identifier from the at least one of the plurality of uplink trigger subframes. As previously noted in the embodiments described above, this identifier may include a running count of uplink trigger subframe, thereby replacing the need for a counter in the medical devices. Additionally or alternatively, the identifier may comprise an address or other identifier of the medical device.

As noted previously, some embodiments may provide for multiple uplink subframes to be communicated between successive uplink trigger subframes. In some embodiments, the medical device may further identify the at least one of the plurality of uplink trigger subframes by analyzing a pair of pulses in the at least one of the plurality of uplink trigger subframes, where the pair of pulses is modulated such that a first pulse has a first duration, and a second pulse has a second ration different than the first duration. As indicated in the embodiment illustrated in FIG. 4, different pulses may have different amplitudes, to help the medical device identify and parse pulse pairs. In some embodiments, analyzing the pair of pulses in the at least one of the plurality of uplink trigger subframes may comprise comparing a ratio of the first duration to the second duration. In some embodiments, determining when to send the uplink subframe may comprise adjusting a local clock of the medical device based on when the least one of the plurality of uplink trigger subframes was received. For example, a medical device may synchronize its LO (or other clock based thereon) based on one an uplink trigger subframes received. Means for performing the functionality at block 620 may comprise, for example, bus 905, processing unit(s) 910, memory 920, and/or other components of a medical implant 130, as illustrated in FIG. 9 and described in more detail below.

At block 630, the medical device sends the uplink subframe. Here, as noted previously, the uplink subframe can be transmitted between successive uplink trigger subframes, each medical device transmitting its respective uplink subframe during its respective time slot during the communication frame. Means for performing the functionality at block 630 may comprise, for example, bus 905, processing unit(s) 910, memory 920, communication interface 930, antenna 935, and/or other components of a medical implant 130, as illustrated in FIG. 9 and described in more detail below.

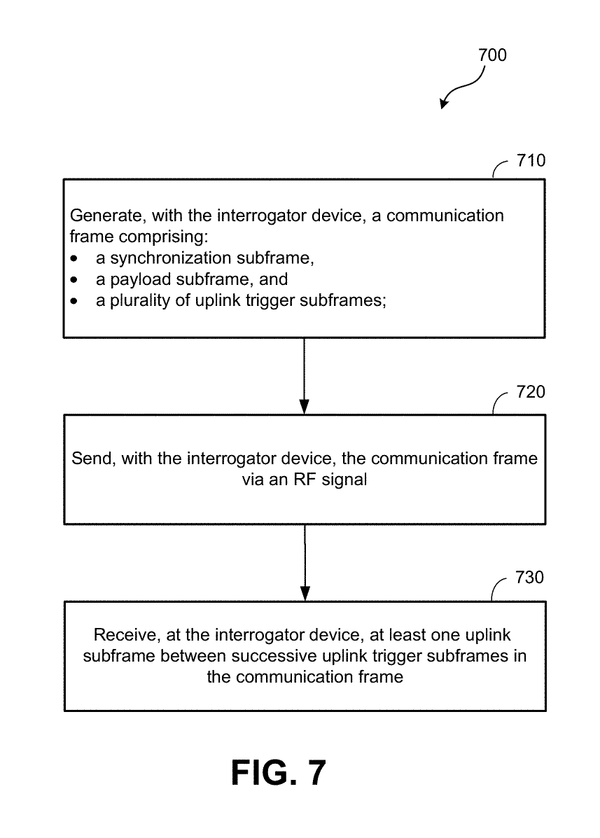

FIG. 7 is a flow diagram of an example method 700 of enabling synchronize wireless communication with an interrogator device, according to an embodiment. The functionality described in one or both blocks illustrated in FIG. 7 may be performed, for example, by an interrogator device in a low-power wireless system, such as an interrogator device 140 of the wireless medical implant system illustrated in FIG. 1. Accordingly, means for performing this functionality may include hardware and/or software components of an interrogator device 140. An example of such hardware and/or software components is illustrated in FIG. 8 and described in more detail below.

The functionality at block 710 comprises generating, with the interrogator device, a communication frame comprising a synchronization subframe, a payload subframe, and a plurality of uplink trigger subframes. Again, the communication frame here may comprise an operating frame as illustrated in FIG. 3, and described in more detail above. And, as noted above, plurality of uplink trigger subframes may be spaced apart in time such that at least one uplink subframe can be transmitted between successive uplink trigger subframes. In some embodiments, the method may further comprise including, in each uplink trigger subframe of the plurality of uplink trigger subframes, and identifier unique the respective uplink trigger subframe. Again, this identifier may comprise an increasing uplink trigger subframe count, an address of a medical implant, and/or some other identifier to uniquely identify the uplink trigger subframe. In some embodiments, the method may further comprise including, in each uplink trigger subframe of the plurality of uplink trigger subframes, a first pair of pulses, where the first pair of pulses uses a first modulation scheme such that a first pulse has a first duration and a second pulse has a second ration different from the first duration. As noted above, the first modulation scheme may be based on a particular ratio between long and short pulses. As such, according to some embodiments, the method may comprise causing a ratio of the first duration to the second ration to exceed a threshold. As noted above, for example, a ratio of the long pulse to the short pulse may exceed a threshold of 6:1, in some embodiments. (This threshold may be larger or smaller, depending on desired functionality. Of course, a lower threshold (e.g., 1:6) may be exceeded if pulse durations are reversed.) In some embodiments, the first modulation scheme is different than a second modulation scheme used to modulate a second pair of pulses in the payload subframe. For example, where the ratio of the duration of long to short pulses in the first modulation scheme may meet or exceed 6:1, the ratio of duration of long to short pulses in the second modulation scheme may be approximately or substantially 2:1. Of course, other undulation schemes may be used. Means for performing the functionality at block 710 may comprise, for example, bus 805, processing unit(s) 810, memory 850, and/or other components of an interrogator device 140, as illustrated in FIG. 8 and described in more detail below.

The functionality at block 720 comprises sending, with the interrogator device, the communication frame via an RF signal. As noted above, the interrogator device may continue to transmit an RF carrier between successive uplink trigger subframes where medical devices (e.g., medical implants) may need the RF carrier for power purposes. Means for performing the functionality at block 720 may comprise, for example, bus 805, processing unit(s) 810, memory 850, communication interface 840, antenna 845, and/or other components of an interrogator device 140, as illustrated in FIG. 8 and described in more detail below.

The functionality at block 730 comprises receiving, at the interrogator device, at least one uplink subframe between successive uplink trigger subframes in the communication frame. As noted above, the interrogator device may continue to transmit an RF carrier between successive uplink trigger subframes where medical devices (e.g., medical implants) may need the RF carrier for power purposes. Each medical device may transmit an uplink subframe during its allocated time slot (between uplink trigger subframes). Means for performing the functionality at block 720 may comprise, for example, bus 805, processing unit(s) 810, memory 850, communication interface 840, antenna 845, and/or other components of an interrogator device 140, as illustrated in FIG. 8 and described in more detail below.

FIG. 8 is a simplified block diagram of an interrogator device 140, according to an embodiment. The interrogator device 140 may comprise a “skin patch” (similar to the interrogator device of FIG. 1) or other device configured to perform one or more of the functions of an interrogator device as described in embodiments herein. FIG. 8 is meant only to provide a generalized illustration of various components, any or all of which may be included or omitted as appropriate. The interrogator device 140 may be configured to execute one or more functions of the methods described herein, such as the method 700 illustrated in FIG. 7. It can be further noted that the interrogator device 140 may be configured to receive measurements from and/or stimulate a body part utilizing one or more medical devices, such as medical implants, with which the interrogator device 140 is in wireless communication, as described in the embodiments above. In some embodiments, the particular measurements taken and/or stimulations may be determined by the interrogator device 140 itself, and/or be determined by another device (such as a medical device, mobile phone, tablet, etc.) with which the interrogator device 140 is in communication. A person of ordinary skill in the art will understand that, for the sake of simplicity, some components (e.g., power source, clock, physical housing, etc.) are not shown.

The interrogator device 140 is shown comprising hardware elements that can be electrically coupled via a bus 805 (or may otherwise be in communication, as appropriate). The hardware elements may include a processing unit(s) 810 which may comprise without limitation one or more general-purpose processors, one or more special-purpose processors (such as digital signal processing (DSP) chips, graphics acceleration processors, application specific integrated circuits (ASICs), and/or the like), and/or other logic, processing structure, or means, which can be configured to perform one or more of the methods described herein.

Depending on desired functionality, the interrogator device 140 also may comprise one or more input devices 820, which may comprise without limitation one or more, touch sensors, buttons, switches, and/or more sophisticated input components, which may provide for user input, which may enable the system to power on, configure operation settings, and/or the like. Output device(s) 830 may comprise, without limitation, light emitting diode (LED)s, speakers, and/or more sophisticated output components, which may enable feedback to a user, such as an indication the implant system has been powered on, is in a particular state, is running low on power, and/or the like.

The interrogator device 140 might also include a communication interface 840 and one or more antennas 845. This communication interface 840 and antenna(s) 845 can enable the interrogator device 140 to communicate with and optionally power the medical implants of the wireless medical implant system. The one or more antennas 845 can be configured to, when powered properly, generate particular signals and/or fields to communicate with and/or power the medical implants, including communicating medical implant selection methods as described herein. As previously indicated, medical implants in some embodiments may communicate using RF backscatter, in which case the interrogator device 140 may transmit an RF carrier signal, modulated by the medical implants during uplink communications.

In some embodiments, the processing unit(s) 810 and/or communication interface 840 (including software and/or firmware executed therewith) may create a communication frame and/or perform amplitude modulation of an RF signal to encode the RF signal with the communication frame, as described herein.

The communication interface 840 may further enable the interrogator device 140 to communicate with one or more devices outside the biological measurement and stimulation system to which the interrogator device 140 belongs, such as a medical device, mobile phone, tablet, etc. In some embodiments, the one or more devices may execute a software application that provides a user interface (e.g., a graphical user interface) for configuring and/or managing the operation of the interrogator device 140.

The communication interface may include connectors and/or other components for wired communications (e.g., universal serial bus (USB) Ethernet, optical, and/or other communication). Additionally, or alternatively, the communication interface 840 and optionally the antenna(s) 845 may be configured to provide wireless communications (e.g., via Bluetooth®, Bluetooth® low energy (BLE), Institute of Electrical and Electronics Engineers (IEEE) 802.11, IEEE 802.15.4 (or ZIGBEE®), Wi-Fi, WiMAX™, cellular communications, infrared, etc.). As such, the communication interface 840 may comprise without limitation a modem, a network card, an infrared communication device, a wireless communication device, and/or a chipset.

The interrogator device 140 may further include and/or be in communication with a memory 850. The memory 850 may comprise, without limitation, local and/or network accessible storage such as optical, magnetic, solid-state storage (e.g., random access memory (“RAM”) and/or a read-only memory (“ROM”)), or any other non-transitory, computer-readable medium. The memory 850 may therefore make the interrogator device 140 can be programmable, flash-updateable, and/or the like. Such storage devices may be configured to implement any appropriate data stores, including without limitation, various file systems, database structures, and/or the like.

The memory 850 of the interrogator device 140 also can comprise software elements (not shown), including an operating system, device drivers, executable libraries, and/or other code, such as one or more application programs, which may comprise computer programs provided by various embodiments, and/or may be designed to implement methods, and/or configure systems, provided by other embodiments, as described herein. For example, one or more procedures described with respect to the functionality discussed above might be implemented as computer code and/or instructions executable by the interrogator device 140 (and/or processing unit(s) 810 of the interrogator device 140). The memory 850 may therefore comprise non-transitory machine-readable media having the instructions and/or computer code embedded therein/thereon.

FIG. 9 is a simplified block diagram of a medical implant 130, according to an embodiment. The medical implant 130 may comprise a “neurograin” (similar to the medical implants 130 of FIG. 1) or other device configured to perform one or more of the functions of a medical implant of a biological measurement and stimulation system as described in embodiments herein. It will be understood that medical devices that are not implanted or implantable, may comprise similar components. FIG. 9 is meant only to provide a generalized illustration of various components, any or all of which may be included or omitted as appropriate. It can be further noted that the medical implant 130 may be configured to take measurements and/or stimulate a body part as directed by an interrogator device 140 using communications such as those described in the embodiments herein. A person of ordinary skill in the art will understand that, for the sake of simplicity, some components (e.g., power source, LO, physical housing, etc.) are not shown. It will be understood that, in most embodiments, hardware and/or software optimizations may be made to help minimize power consumption.

The medical implant 130 is shown comprising hardware elements that can be electrically coupled via a bus 905, or may otherwise be in communication, as appropriate. The hardware elements may include a processing unit(s) 910 which may comprise without limitation one or more general-purpose processors, one or more special-purpose processors (e.g., microprocessors), and/or other logic, processing structure, or means, which can be configured to perform one or more of the methods described herein. As a person of ordinary skill in the art will appreciate, the processing unit(s) 910, may further include one or more splicers, counters, and/or other circuitry as described herein (and/or may implement the functions of such circuitry and software) for processing incoming RF signal. Additionally or alternatively, such circuitry (or software) he be implemented in the communication interface 930, described in more detail below.

The medical implant 130 may further include and/or be in communication with a memory 920. As with other components of the medical implant 130, the memory 920 may be optimized for minimum power consumption. In some embodiments, the memory 920 may be incorporated into the processing unit(s) 910. Depending on desired functionality, the memory (which can include a non-transitory computer-readable medium, such as a magnetic, optical, or solid-state medium) may include computer code and/or instructions executable by the processing unit(s) 910 to perform one or more functions described in the embodiments herein.

A communication interface 930 and antenna(s) 935 can enable the medical implant 130 to wirelessly communicate the interrogator device, as described herein. The antenna(s) 935 may comprise a coiled or other antenna configured to draw power from communications and/or other signals or fields generated by the interrogator device, powering the medical implant 130. In some embodiments, the medical implant 130 may further include an energy storage medium (e.g., a battery, capacitor, etc.) to store energy captured by the antenna(s) 935. In some embodiments, the communication interface 930 and antenna(s) 935 may be configured to the interrogator device using RF backscatter, as noted above.

The stimulator(s) 940 of the medical implant 130 can enable the medical implant 130 to provide stimulation to a body part (e.g., biological tissue) in which the medical implant 130 is implanted. As such, the stimulator(s) 940 may comprise an electrode, LED, and/or other component configured to provide electrical, optical, and/or other stimulation. The processing unit(s) 910 may control the operation of the stimulator(s) 940, and may therefore control the timing, amplitude, and/or other stimulation provided by the stimulator(s) 940.

The sensor(s) 950 may comprise one or more sensors configured to receive input from a body part (e.g., biological tissue), in which the medical implant 130 is implanted. Sensors may therefore be configured to sense electrical impulses, pressure, temperature, light, conductivity/resistivity, and/or other aspects of a body part. As described herein, embodiments may enable medical implant 130 to provide this information, via the communication interface 930, to an interrogator. Depending on desired functionality, information received by the sensor(s) 950 may be encrypted, compressed, and/or otherwise processed before it is transmitted via the communication interface 930.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

It has proven convenient at times, principally for reasons of common usage, to refer to such signals as bits, information, values, elements, symbols, characters, variables, terms, numbers, numerals, or the like. It should be understood, however, that all of these or similar terms are to be associated with appropriate physical quantities and are merely convenient labels. Unless specifically stated otherwise, as is apparent from the discussion above, it is appreciated that throughout this Specification discussions utilizing terms such as “processing,” “computing,” “calculating,” “determining,” “ascertaining,” “identifying,” “associating,” “measuring,” “performing,” or the like refer to actions or processes of a specific apparatus, such as a special purpose computer or a similar special purpose electronic computing device. In the context of this Specification, therefore, a special purpose computer or a similar special purpose electronic computing device is capable of manipulating or transforming signals, typically represented as physical electronic, electrical, or magnetic quantities within memories, registers, or other information storage devices, transmission devices, or display devices of the special purpose computer or similar special purpose electronic computing device.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AA, AAB, AABBCCC, etc.

Having described several embodiments, various modifications, alternative constructions, and equivalents may be used without departing from the spirit of the disclosure. For example, the above elements may merely be a component of a larger system, wherein other rules may take precedence over or otherwise modify the application of the invention. Also, a number of steps may be undertaken before, during, or after the above elements are considered. Accordingly, the above description does not limit the scope of the disclosure.

What is claimed is:

1. A medical device comprising:
a communication interface configured to receive a communication frame transmitted via a radio frequency (RF) signal and comprising:
a synchronization subframe,
a payload subframe, and
a plurality of uplink trigger subframes spaced apart such that at least one uplink subframe can be transmitted between successive uplink trigger subframes;
a processing unit communicatively coupled with the communication interface and configured to:
determine when to send an uplink subframe via the communication interface based, at least in part, on when at least one of the plurality of uplink trigger subframes was received; and
send, via the communication interface, the uplink subframe.

2. The medical device of claim 1, wherein the processing unit is configured to determine when to send the uplink subframe by counting a number of uplink trigger subframes of the plurality of uplink trigger subframes.

3. The medical device of claim 1, wherein the processing unit is configured to determine when to send the uplink subframe by obtaining an identifier from the at least one of the plurality of uplink trigger subframes.

4. The medical device of claim 1, further comprising a local clock, wherein the processing unit is configured to determine when to send the uplink subframe by adjusting the local clock based on when the at least one of the plurality of uplink trigger subframes was received.

5. The medical device of claim 1, wherein the processing unit is further configured to identify the at least one of the plurality of uplink trigger subframes by analyzing a pair of pulses in the at least one of the plurality of uplink trigger subframes, wherein the pair of pulses is modulated such that:
a first pulse has a first duration, and
a second pulse has a second duration different than the first duration.

6. The medical device of claim 5, the processing unit is configured to analyze the pair of pulses in the at least one of the plurality of uplink trigger subframes by comparing a ratio of the first duration to the second duration.

7. The medical device of claim 1, wherein the processing unit is configured to send the uplink subframe between successive uplink trigger subframes of the plurality of uplink trigger subframes.

8. An interrogator device comprising:
a processing unit configured to generate a communication frame comprising:
a synchronization subframe,
a payload subframe, and
a plurality of uplink trigger subframes;
a communication interface communicatively coupled with the processing unit and configured to:
send the communication frame via a radio frequency (RF) signal; and
receive at least one uplink frame between successive uplink trigger subframes in the communication frame.

9. The interrogator device of claim 8, wherein the processing unit is further configured to include, in each uplink trigger subframe of the plurality of uplink trigger subframes, an identifier unique to the respective uplink trigger subframe.

10. The interrogator device of claim 8, wherein the processing unit is further configured to include in each uplink trigger subframe of the plurality of uplink trigger subframe a first pair of pulses, wherein the first pair of pulses uses a first modulation scheme such that:
a first pulse has a first duration, and
a second pulse has a second duration different than the first duration.

11. The interrogator device of claim 10, wherein the processing unit is further configured to cause a ratio of the first duration to the second duration to exceed a threshold.

12. The interrogator device of claim 10, wherein the processing unit is configured to use a second modulation scheme to modulate a second pair of pulses in the payload subframe, wherein the second modulation scheme is different than the first modulation scheme.

13. A method of synchronizing wireless communication at a medical device, the method comprising:
receiving, at the medical device, a communication frame transmitted via a radio frequency (RF) signal and comprising:
a synchronization subframe,
a payload subframe, and
a plurality of uplink trigger subframes spaced apart such that at least one uplink subframe can be transmitted between successive uplink trigger subframes;
determining, with the medical device, when to send an uplink subframe based, at least in part, on when at least one of the plurality of uplink trigger subframes was received; and
sending, with the medical device, the uplink subframe.

14. The method of claim 13, wherein determining when to send the uplink subframe comprises counting a number of uplink trigger subframes of the plurality of uplink trigger subframes.

15. The method of claim 13, wherein determining when to send the uplink subframe comprises obtaining an identifier from the at least one of the plurality of uplink trigger subframes.

16. The method of claim 13, wherein determining when to send the uplink subframe comprises adjusting a local clock of the medical device based on when the at least one of the plurality of uplink trigger subframes was received.

17. The method of claim 13, further comprising identifying the at least one of the plurality of uplink trigger subframes by analyzing a pair of pulses in the at least one of the plurality of uplink trigger subframes, wherein the pair of pulses is modulated such that:
a first pulse has a first duration, and
a second pulse has a second duration different than the first duration.

18. The method of claim 17, wherein analyzing the pair of pulses in the at least one of the plurality of uplink trigger subframes comprises comparing a ratio of the first duration to the second duration.

19. The method of claim 13, wherein sending the uplink subframe comprises sending the uplink subframe between successive uplink trigger subframes of the plurality of uplink trigger subframes.

20. A method of enabling synchronized wireless communication with a interrogator device, the method comprising:
generating, with the interrogator device, a communication frame comprising:
a synchronization subframe,
a payload subframe, and
a plurality of uplink trigger subframes;
sending, with the interrogator device, the communication frame via a radio frequency (RF) signal; and
receiving, at the interrogator device, at least one uplink frame between successive uplink trigger subframes in the communication frame.

21. The method of claim 20, further comprising including, in each uplink trigger subframe of the plurality of uplink trigger subframes, an identifier unique to the respective uplink trigger subframe.

22. The method of claim 20, further comprising including in each uplink trigger subframe of the plurality of uplink trigger subframe a first pair of pulses, wherein the first pair of pulses uses a first modulation scheme such that:
a first pulse has a first duration, and
a second pulse has a second duration different than the first duration.

23. The method of claim 22, further comprising causing a ratio of the first duration to the second duration to exceed a threshold.

24. The method of claim 22, wherein the first modulation scheme is different than a second modulation scheme used to modulate a second pair of pulses in the payload subframe.
